(19) United States
(12) Patent Application Publication
(10) Pub. No.: US 2008/0262346 A1
(43) Pub. Date: Oct. 23, 2008

(54) Method And Apparatus For Blood Vessel Parameter Determinations

(76) Inventors: Tsuriel Assis, Rehovot (IL); Asher Kuper, Herzliya (IL); Michael Zarkh, Givat Shmuel (IL); Moshe Klaiman, Gadera (IL)

Correspondence Address: LADAS & PARRY, 5670 WILSHIRE BOULEVARD, SUITE 2100, LOS ANGELES, CA, 90036-5679, US

(21) Appl. No.: 11/792,688

(22) PCT Filed: Mar. 3, 2006
(86) PCT No.: PCT/IL05/00788

§ 371 Date: Feb. 21, 2008

(30) Foreign Application Priority Data
Dec. 8, 2004 (IL) 165636
Feb. 13, 2005 (IL) 2005/000184

(51) Int. Cl. A61B 5/0275; A61B 6/00

(52) U.S. Cl. 600/431; 600/504

(57) ABSTRACT

An apparatus and method for the determination of the flow, the coronary reserve and relative coronary reserve of a specific coronary artery. The apparatus and method employ a three-dimensional model (50) providing the volume of a segment of the artery at a plurality of points in time, and disclose various options (56) for determining the arterial flow through the artery segment during one or more heart beat cycles or parts thereof. The determination of the coronary reserve (62) and relative coronary reserve (64) follow from the volume (54) and the flow through the artery. Alternatively, the coronary reserve is determined directly from a velocity profile relating to one or more fixed artery segments and the three-dimensional model.

### BACKGROUND OF THE INVENTION

1. Field of the Invention

The present invention relates to blood flow diagnosis in general, and to a method and system for determining the coronary reserve and the relative coronary reserve, in particular.

2. Discussion of the Related Art

The coronary velocity reserve (CVR) also known as the coronary flow reserve (CFR), and generally referenced hereinafter as coronary reserve, is defined to be the ratio between the maximal and the resting coronary blood flow. The maximal coronary blood velocity occurs at times of highly intensive exercise, or it is induced when the subject is injected with coronary vasodilator such as adenosine. The coronary reserve represents the ability of the coronary arteries to supply the excess blood needed to comply with the excessive pumping requirements, relatively to normal (resting) conditions. The normal coronary reserve is greater than 3.0 and in some individuals is greater than 5.0. When the CFR is impaired (values equal to or less than 2.5) the coronary arteries are unable to supply the excess required amounts of blood. Reduced CFR is often associated with angina pectoris, diabetes mellitus, systemic sclerosis, Coronary Syndrome X and other clinical conditions. Determining the CFR is also valuable when assessing the severity of a stenosis in a coronary artery, since there are cases when a stenosis is found, but is not the sole cause of ischemia. Therefore, prior to performing a risky and expensive medical operation, determining the CFR should be considered. Flow measurement is also useful for predicting long-term success of treatment and comparison of efficacy of various treatments. Some currently available techniques for measuring the CFR include invasive methods such as a Doppler catheter, or a pressure wire, which follows a leading catheter that emits the contrast agent. The Doppler catheter or pressure wire methods have several disadvantages. Extra intervention procedures are required, which makes the Doppler catheter or the pressure wire methods more costly, more invasive, and therefore more risky. In addition, the extra wire or catheter affect the flow itself and therefore impair the measured flow. Yet another drawback of the Doppler catheter and the pressure wire is that the catheter or wire must be accurately aligned, otherwise the results are impaired. Another method for measuring the CFR involves Magnetic Resonance (MR). This procedure is used for diagnosis only while the catheterization procedures are used for therapeutic purposes as well. The MR method is also costly and physicians are reluctant to use it. Another method for measuring the CFR is the Digital Subtraction Analysis (DSA). When using the DSA method, the flow is evaluated directly from the angiograms, by measuring the change in the gray level in a specified region, caused by the spreading of the contrast agent. This method uses the relation between the gray level and the volume of the contrast agent (as in densitometry) to calculate the flow directly from the change in gray level between successive frames. A limitation of the DSA methods is the difficulty in calibrating the relation between the gray level and the volume. This relation depends on imaging conditions such as the X-ray energy, the extent of magnification, the distance between the source image and the receptor (SID), or others, and contrast material parameters. There are too many parameters and not enough available information to accurately determine the CFR. Another drawback of the DSA method originates from the area captured in the x-ray. Since this area is substantially larger than the area of the artery, the error calculations are mush larger, which again harms the measurements.

Yet another method for evaluating the CFR is the Contrast Propagation Algorithm (CPA). When using the CPA method, the flow is determined by observing the propagation of the haze. The limitations of the CPA are that it is a difficult task to follow the haze, due to its indistinct nature, and that it is required to know the exact structure of the artery in order to take into account the 3D geometry. Therefore, this method is hard to execute and suffers form inaccuracies.

A parameter related to the coronary flow is the TIMI Flow Grading, which is a qualitative assessment of dye washout during contrast angiography. Using the TIMI flow grading for assessing the coronary reserve is qualitative and subjective, and therefore does not provide accurate measurements.

There is therefore a need for a relatively cost effective, easy to use, accurate and reliable method and system for measuring the CFR of a subject and other flow related measurements. It is desirable that the method will provide accurate results for flow measurement at different times during the cycle of the heart beat, and will incorporate, as a preferred embodiment, the full heart beat cycle for providing accurate average results. It is also desirable that the method can be used during the catheterization, or at a later time, and will not imply extra invasiveness beyond a standard catheterization.

### SUMMARY OF THE PRESENT INVENTION

It is an object of the present invention to provide a novel method and apparatus for the determination of coronary reserve and relative coronary reserve of a specific coronary artery segment and other flow related measurements. In accordance with the present invention, there is thus provided a method for determining the arterial reserve of a subject having a blood flow of a velocity below or equal to a maximal velocity value, the method comprising the following steps: receiving one or more first models, each model representing one or more substantially fixed segments of one or more arteries of the subject at a plurality of points in time associated with and during one or more parts of one or more first heart beat cycles, the artery segment having a proximal cross section and a distal cross section; injecting contrast agent into the artery of the subject, when the subject is in a non-hyperemic state, the contrast agent is injected at an injection area having a proximal cross section and having a distance from the substantially fixed segment; determining one or more first parameters from one or more first angiograms representing the substantially fixed artery segment, said first angiogram taken form a projection angle; injecting the subject with substance that simulates hyperemia; receiving one or more second models, representing the substantially fixed segment of the artery of the subject at a plurality of points in time associated with and during one or more parts of one or mode second heart beat cycles; injecting the contrast agent to the artery of the subject, said subject being in a hyperemic state, said contrast agent is injected at the injection area; determining one or more second parameters from one or more second angiograms representing the substantially fixed segment, said second angiogram taken form a projection angle; and determining the arterial reserve as the ratio between one of the first parameters or a combination thereof and one of the second parameters or a combination thereof. The first parameter can be a ratio between the maximal velocity value of blood within the artery segment when the subject is at a hyperemic state and the distance between the substantially fixed segment and the injection area. The second parameter can be a ratio between the maximal velocity value of blood within the artery segment when the subject is at a non-hyperemic state and the distance of the artery segment from the injection area. Within the method, determination of the first parameters can comprise the steps of: determining from the first angiogram taken at a predetermined projection angle and the first model, a density curve for the artery segment; obtaining a velocity profile of the blood flow within the artery; performing curve fitting for the density curve to determine the first parameter. Within the method, determination of the second parameter can comprise the steps of: determining from the second angiogram taken at a predetermined projection angle and second model, a density curve for the at least one artery segment; obtaining a velocity profile of the blood flow within the artery; performing curve fitting for the density curve to determine the second parameter. The distance between the injection area and the substantially fixed segment can be the distance between the distal cross section of the injection area and a cross section of the substantially fixed artery segment located at equal distances from the proximal cross section and from the distal cross section of the substantially fixed artery segment. The method can further comprise the step of creating the first or the second models of the artery of the subject. The first or the second models can be three-dimensional models. The method can further comprise the step of determining the projection angle and the volumes of the fixed segment of the artery of the subject at a plurality of points in time associated with and during the one or more parts of the first heart beat cycle, from the first model and the step of determining the projection angle and the volumes of the fixed segment of the at least one artery of the subject at a plurality of points in time associated with and during the one or more parts of the second heart beat cycle, from the second model. The method can further comprise the step of compensating for the non-perpendicularity of the substantially fixed segment of the artery of the subject. The method can further comprise the step of registering the first angiogram with the first model or the second angiogram with the second model. The method can further comprise a step of determining TIMI grades from local gray level curves in multiple points of the artery. The method can further comprise the step of determining a relative arterial reserve as the ratio between the arterial reserve determined for a first artery segment and the arterial reserve determined for a second artery segment. The first artery segment can be diseased or suspect as being diseased and the second artery segment can be healthy. The arterial reserve can be arterial coronary reserve. The contrast agent injection can performed during the systole of the subject, or continuously throughout an integer number of heart beat cycles of the subject. The contrast agent can be injected radially.

Another aspect of the present invention relates to a method for determining the blood flow output of a subject having a blood flow having velocity values below or equal to a maximal velocity, the method comprising the steps of: receiving one or more models, representing one or more substantially fixed segments of one or more arteries of the subject at a plurality of points in time associated with and during one or more parts of one or more heart beat cycles; injecting contrast agent into the artery of the subject at an injection area having a distance from the substantially fixed segment; determining from one or more angiograms taken at a projection angle and the model, a density curve for the substantially fixed segment; obtaining a velocity profile of the blood flow within the substantially fixed segment; performing curve fitting for the density curve to determine one or more parameters; substituting the at parameters in the velocity profile to determine velocity values; and integrating the velocity values over a cross section of the substantially fixed segment to obtain the arterial segment output values of the blood flow within the substantially fixed segment of the artery. Within the method, the parameter can be the ratio between the maximal velocity of blood within the substantially fixed segment and the distance of the substantially fixed segment from the injection point. Within the method, the distance between the injection area and the substantially fixed segment is the distance between the distal cross section of the injection area and a cross section of the substantially fixed artery segment located at equal distances from the proximal cross section and from the distal cross section of the substantially fixed artery segment. The method can further comprise the step of creating the model of the substantially fixed segment of the artery of the subject. The method can further comprise the step of determining the projection angle from the model, or the step of compensating for the non-perpendicularity of the substantially fixed segment of the artery of the subject. The method can further comprise the step of registering the angiograms with the model. Within the method, the model can be a three-dimensional model. The method can further comprise a step of determining TIMI grades from local gray level curves in multiple points of the artery.

Yet another aspect of the present invention relates to a method for determining the arterial reserve of a subject, the method comprising the following steps: receiving one or more first models, representing two or more substantially fixed segments of one or more arteries of the subject at a plurality of points in time associated with and during one or more parts of one or more first heart beat cycles; injecting contrast agent into the artery of the subject, said contrast agent is is delivered to two or more substantially fixed segments being in a non-hyperemic state; determining from one or more first angiograms taken at a predetermined projection angle, a first set of density curves, one density curve for each of the two or more substantially fixed segment; injecting the subject with substance that simulates hyperemia; receiving one or more second models, representing the two or more substantially fixed segments of the artery being in a hyperemic state, at times corresponding to a plurality of points in time associated with one or more parts of one or more second heart beat cycles; injecting the contrast agent to the artery of the subject, said contrast agent is delivered to the two or more substantially fixed segments being in a hyperemic state; determining from one or more second angiograms taken at a predetermined projection angle, a second set of density curves, one density curve for each of the two or more substantially fixed segment. The method can further comprise the steps of: determining a first time shift between a first density curve taken from the first set of density curves and a second density curve taken from the second set of density curves, the first and the second density curves corresponding to a first substantially fixed segment; determining a second time shift between a third density curve taken from the first set of density curves and a fourth density curve taken from the second set of density curves, the third and the fourth density curves corresponding to the second substantially fixed segment; and determining the arterial reserve as the ratio between the first time shift and the second time shift. The method can further comprise the steps of: shrinking the first set of density curves in a shrinking factor so that maximal similarity occurs between the shrunk first set and the second set; and determining the arterial reserve as the shrinking factor. Alternatively, the method can further comprise the steps of: stretching the second set of density curves in a stretching factor so that maximal similarity occurs between the first set and the stretched second set; and determining the arterial reserve as the stretching factor.

Yet another aspect of the disclosed invention relates to an apparatus for determining the arterial reserve of a subject from two or more images, the apparatus comprises: a component for receiving a model of one or more substantially fixed segment of one or more arteries, the segment having a volume; a gray level extraction component for extracting the gray level representing the material filling rate and diminishing rate along the at least one artery; a density curve construction component for constructing a density curve associated with the substantially fixed segment from the gray levels; a curve fitting component for determining a parameter associated with the density curve or a part thereof; and an enhanced artery reserve component for determining the arterial reserve as a ratio between a first parameter associated with a first density curve associated with the substantially fixed segment being in a hyperemic state and a second parameter associated with a second density curve associated the substantially fixed segment being in a non-hyperemic state. The apparatus can further comprise a segment volume component for determining the projection angle or the volume of the substantially fixed segment of an at least one artery from the model. The apparatus can further comprise a relative arterial reserve component for determining the relative arterial reserve between the first artery and a second artery, said relative arterial reserve being the ratio between the arterial reserve of the first artery and the arterial reserve of the second artery. The apparatus can further comprise one or more image acquiring devices. The apparatus can further comprise a device for transferring images acquired by an image acquiring device to a processing unit, the processing unit comprises one or more input and one or more output devices for receiving input and presenting output to a user. The apparatus can further comprise a storage device for storing the images or the determined arterial reserve values.

Yet another aspect of the disclosed invention relates an apparatus for determining the arterial flow of a subject from two or more images, the apparatus comprising: a component for receiving a model and volumes of one or more substantially fixed segment of one or more arteries; a gray level extraction component for extracting gray level representing the material filling rate and diminishing rate along the artery; a density curve construction component for constructing a density curve from the gray levels; a curve fitting component for fitting a curve to the density curve or a part thereof; a fractional contrast material volume component for determining the artery segment output during the one or more parts of the one or more heart beat cycles of the artery. The apparatus can further comprise a segment volume component for determining the volume of one or more substantially fixed segments of one or more arteries.

### BRIEF DESCRIPTION OF THE DRAWINGS

The present invention will be understood and appreciated more fully from the following detailed description taken in conjunction with the drawings in which:

FIG. 1 is a schematic illustration of an exemplary environment in which one embodiment of the proposed invention is used;

FIG. 2 is a schematic block diagram of the software or firmware computational components of the proposed invention, in accordance with a preferred embodiment of the disclosed invention;

FIG. 3 is a schematic flow chart of the proposed method for measuring the coronary reserve and the relative coronary reserve, in accordance with a preferred embodiment of the disclosed invention;

FIG. 4 is a graph showing the gray level representing the contrast agent concentration in a specific location in an artery, in accordance with a preferred embodiment of the disclosed invention;

FIG. 5 is a graph showing the gray level as representing the concentration of contrast agent as a function of time and location, in accordance with a preferred embodiment of the disclosed invention;

FIG. 6 is a graph illustrating the analysis of the graph shown in FIG. 5, in accordance with a preferred embodiment of the disclosed invention;

FIG. 7 is a schematic block diagram of the software or firmware computational components of an alternative preferred embodiment of the disclosed invention;

FIG. 8 is a schematic flow chart of the proposed method for measuring the coronary reserve and the relative coronary reserve, in accordance with an alternative preferred embodiment of the disclosed invention;

FIG. 9A is an illustration of an artery segment during contrast material injection, in accordance with a preferred embodiment of the disclosed invention;

FIG. 9B is an illustration of an artery segment after a contrast material injection, in accordance with a preferred embodiment of the disclosed invention;

FIG. 10 is a density curve, in accordance with a preferred embodiment of the disclosed invention;

FIG. 11 is a density curve of a pulsatile flow, in accordance with a preferred embodiment of the disclosed invention;

FIG. 12A shows an artery with two regions of interest, in accordance with a preferred embodiment of the disclosed invention;

FIG. 12B shows two density curves at a non-hyperemic state, in accordance with a preferred embodiment of the disclosed invention; and

FIG. 12C shows two density curves at a hyperemic state, in accordance with a preferred embodiment of the disclosed invention.

### DETAILED DESCRIPTION OF THE PREFERRED EMBODIMENT

A new and novel apparatus and method for the determination of coronary reserve and relative coronary reserve and other flow related measurements of a specific coronary artery is disclosed. The proposed method and apparatus first determines the 3D model, including the volume of a fixed segment of an artery at multiple points of time throughout a heart beat cycle. Alternatively, the method uses extrapolation of the 3D model, including the associated volumes from one time segment to another. In yet another alternative, the 3D model and is volumes are received from another source, such as CardiOp by Paieon of Rosh Ha'ayin, Israel. Then, using a contrast agent injected to the subject, the apparatus measures the rate at which the injected material fills the segment of the artery examined. The filling rate and the volume of the segment of the artery examined at the same point in time relatively to the heart beat cycle are used to determine the velocity of the blood flow at that point in time. The velocity of the blood flow over the heart beat cycle is measured in two preferred embodiments. In one preferred embodiment of the present invention, the velocity is calculated by analyzing the gray level through the whole artery segment between two consequent images, and then integrating and averaging over one or more full heart beat cycles. In the second preferred embodiment, the velocity is measured using multiple points along the artery segment, analyzing the local change over time of the gray level. Using the velocity, the process yields the coronary flow output at resting conditions. Then, the subject is injected with coronary vasodilator such as adenosine, simulating hyperemia, and the process is generally repeated. The 3D model and volume determination step can be skipped, since the volume of the large arteries might change insignificantly as the result of the injection. Additionally, since the heart beat cycle might be shorted after the injection, it is possible to down sample the series of volumes. The coronary reserve is then calculated as the ratio between the coronary output in hyperemia, and the arterial output under resting conditions. This method can be applied to any artery in the body of the subject, including the coronary arteries. In accordance with another embodiment of the present invention, in order to determine the relative coronary reserve, the whole process is repeated for a second coronary artery, which is known to be healthy, and a coronary reserve is determined for the second artery. The ratio between the two coronary reserves is the relative coronary reserve.

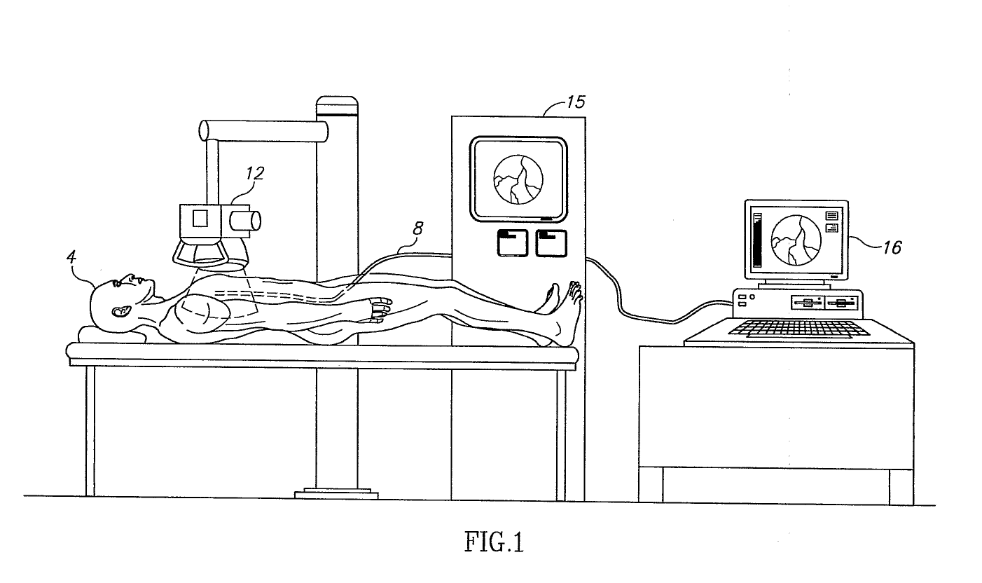

Referring now to FIG. 1 depicting an exemplary environment in which one preferred embodiment of the present invention is used. As shown in the figure in question, a patient subject 4 is lying down in an operating room, being catheterized. The catheter 8 is inserted into the subject's body at the groin, or at another location and is moved through arteries to the area of the heart. Any known methods of catheterized can be used in conjunction with the present invention. At the physician's discretion, the catheter emits a certain amount of contrast agent into an artery of the patient. A computerized angiography device 12 directed at the tip of the catheter is then activated to produce one or more angiograms of the field of view. The angiograms depict the areas where the contrast agent is present in various levels of gray color, in accordance with the concentration of the agent. The resulting images depict the arteries that contain contrast agent. The higher the imaging rate, the better the accuracy of the proposed method. In accordance with a preferred embodiment, the angiograms are transferred from the catheterization work station 15, which is a standard component of a catheterization laboratory (cath-lab) to a work station 16 which uses the images and data transferred from the catheterization work station 15. The work station 16 displays information to the operator of the apparatus and sets the parameters for injecting the patient with contrast agent during the flow analysis. Once the angiograms have been taken, the patient himself does not have to be present at the site. The flow reserve calculation can be carried on immediately after the angiograms have been taken or at a later time, such as a predetermined time set by the user of the apparatus of the present invention. In other preferred embodiments of the present invention the catheterization work station 15 also comprises the apparatus of the present invention as an integrated capability. The work station 16 is preferably a computing platform, such as a personal computer, a mainframe computer, or any other type of computing platform that is provisioned with a memory device (not shown), a CPU or microprocessor device (not shown), and several I/O ports (not shown). Alternatively, the work station 16 can be a DSP chip (not shown), an ASIC device (not shown) storing the commands and data necessary to execute the methods of the present invention, or the like. The work station 16 can further include a storage device (not shown), storing the coronary reserve determination application. The coronary reserve determination application is a set of logically inter-related computer programs or components of computer components and associated data structures that interact to determine the coronary reserve and the relative coronary reserve from the angiograms. The angiograms are preferably transferred to the work station 16 via a transferring device such as a pre-defined I/O port (not shown), DICOM-implementing interface, or analog lines. The angiograms are processed by an apparatus of the present invention. The work station 16 has input and output devices, preferably a keyboard, a mouse and a display where the physician or another stuff member can view or manipulate the angiograms and the products of the application. Alternatively, work station 16 delivers the output to another system.

In an alternative embodiment of the present invention, the coronary reserve is calculated offline, based on angiograms that were taken at an earlier time and possibly at a different location. For example, where the apparatus of the present invention receives angiograms taken from a patient located in a remote area, such as in a different building, or a different, city, state, country and then transmitted at the same time or later, such as even after the patient has recovered or his condition has worsen, via communication lines to the location where the apparatus of the present invention is located, then the angiograms can be analyzed and processed by the apparatus at such time and in other location, effectively allowing the use of the apparatus at any time or location irrespective of the location of the patient, or the time at which the angiograms were taken. Thus, no interaction with the patient is required beyond taking the angiograms.

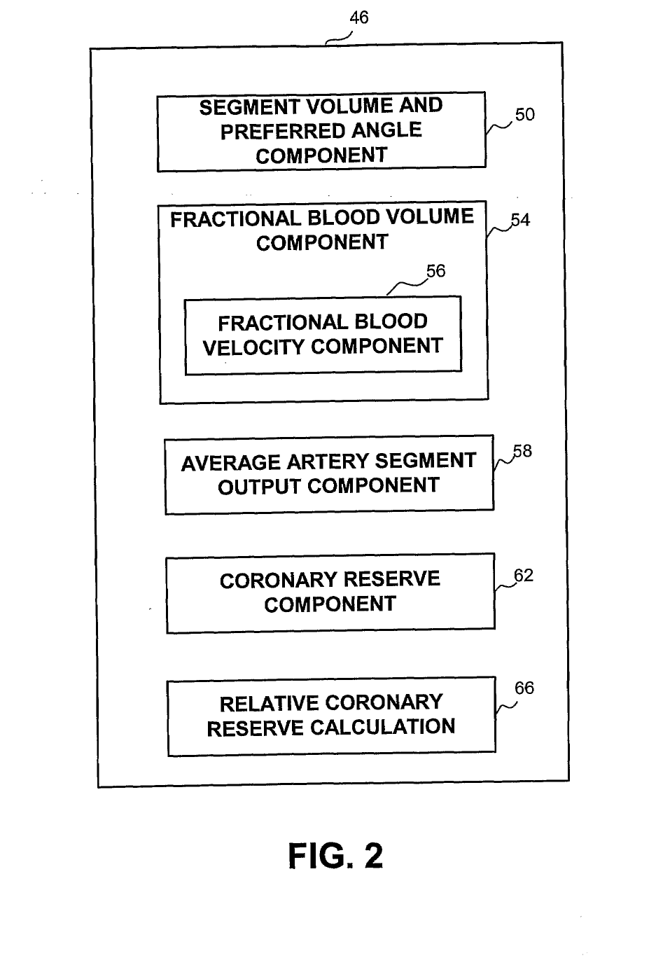

Referring now to FIG. 2, showing the main software or firmware components of the coronary reserve determination application, generally referred to as 46. The segment volume and preferred angle component 50 is a computer program or part of a program, such as CardiOp by Paieon of Rosh Ha'ayin, Israel, when used frame by frame or when used for 3D modeling and volume extrapolation. The segment volume and preferred angle component 50 takes as input a series of images of a fixed segment of an artery, taken at n equally spaced points in time throughout a heart beat cycle, computes a three-dimensional model of the artery segment, and from that model determines a series of numbers:
{Vᵢ}

where i denotes the index of the relevant point in time, and Vᵢ. is the volume of the artery segment at time i. The segment volume and preferred angle component 50 also outputs an imaging direction in which the artery segment should be imaged. This direction minimizes the foreshortening of the artery segment of interest, and is therefore substantially perpendicular to the artery segment. Since the artery segment is generally not straight, the three-dimensional model of the artery segment uses also for compensating for the local non-perpendicularity between the imaging view and the artery segment. Alternatively, step 50 can be skipped when the three-dimensional model, outputs {Vᵢ} and the projection angle are provided by an external source.

The fractional blood volume component 54 receives as input a series of images taken immediately after injecting a contrast agent into the artery. The images are taken substantially perpendicularly to the artery, using the angle calculated by the segment volume and preferred angle for reserve analysis images component 50, so that the average gray level of the internal part of the artery in the image represents the amount of contrast agent present at the artery segment at that instance. The output of the fractional blood volume calculating component is the blood volume that passes through the artery segment between time i−1 and time i, for all i between 1 and n. The registration of images taken at the different stages of the process is enabled by using the three-dimensional model of the artery segment, created or received in step 50. Since the 3D model of the artery and its imaging geometry are available, the registration task should be performed on lateral shift only, thus minimizing registration errors.

The fractional velocity component 56, determines the average velocity of the blood flow at each time segment i. Two preferred methods for velocity measure are detailed hereinafter.

In one preferred method associated with the present invention, the fractional velocity component 56 takes as input the change in the average gray level of the pixels depicting the inside of the artery in images taken at the i−1 and the i-th points of time, representing the velocity of the contrast agent flowing through the artery segment at that time (the higher the change in the gray level—the higher the velocity of the material, and vice versa). Therefore, the time derivative of the gray level represents the velocity of the material through the artery at that time. Using the volume of the artery segment, Vᵢ, at the same point of time relatively to the heart beat cycle, and the velocity of the contrast material yields the volume of the blood passing in the artery segment during each time slot. When using this embodiment, the amount and rate of the injected contrast material should not cause the gray level image to reach saturation. The gray level over all the pixels of the cross section of the artery at location l along the artery segment in image i is denoted by Gᵢ*(l). The projected gray level at every point l along the artery segment in image i, which takes into account the angle between the line of sight of the angiogram and the local direction of the artery, τ, is denoted by g(Gᵢ*(l),τ). For example, the function g can take the form of:

g(Gᵢ*(l),τ)=Gᵢ*(l)*sin(τ)

Then, the projected gray level over the whole artery segment is calculated by the formula:

$$Gi = {\int }_{L}^{ } g ( {G}_{i}^{*} ( l ) , τ ) d l$$

The difference between the gray levels of two consecutive images, ΔGi, is therefore:

ΔGi=Gi−Gᵢ₋₁

The linear approximation of the time derivative of the gray level,

$$\frac{Δ Gi}{Δ t}$$

where Δt is the duration of the i-th time segment, represents the velocity of the contrast agent along the artery segment. However, to switch from gray level change to velocity, this ratio should be calibrated using a predetermined function

$$f ( \frac{Δ Gi}{Δ t} , Gi ) .$$

When the contrast agent leaves the artery segment, the change in the gray level is negative. However, the velocity of the blood is still positive. This sign inversion is also taken care of by the function ƒ. Therefore,

$$Si = f ( \frac{Δ Gi}{Δ t} , Gi )$$

where Si is the average velocity of the flow in the artery segment during the i-th time slot.

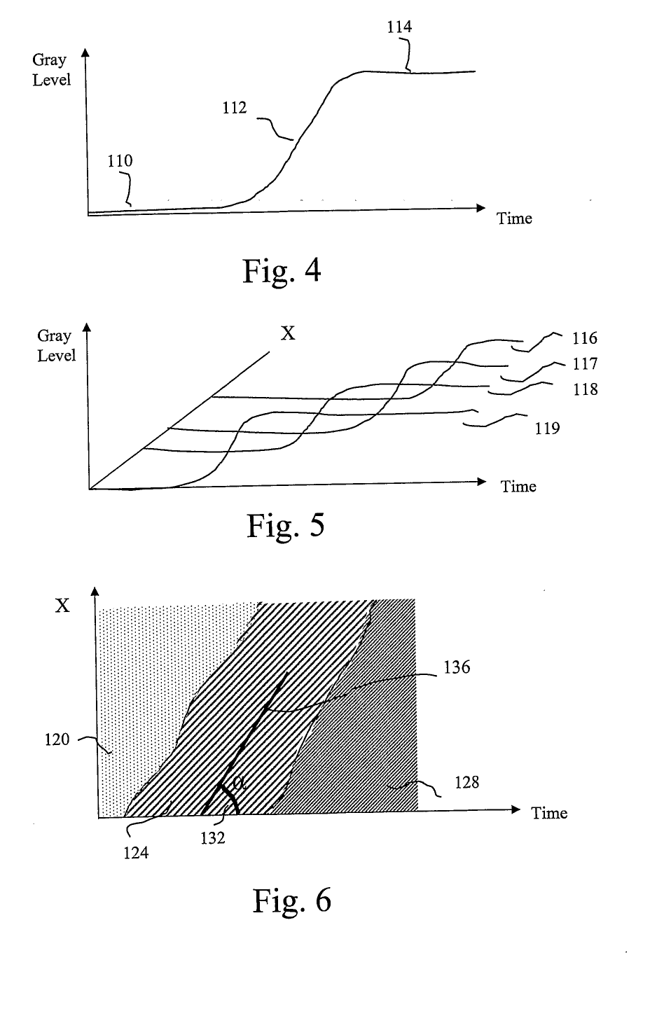

Referring now to FIG. 4, showing an alternative embodiment for the fractional velocity component 56 of FIG. 2. FIG. 4 depicts the gray level at a fixed point x at the artery segment after injecting contrast agent, as a function of time. On the left hand side 110 of the graph of FIG. 4 the gray level depicted is low or non existent, representing a state where contrast agent is not present in the relevant segment to be examined. When injecting the contrast agent to the artery, the contrast agent flows through the arteries and reaches the relevant segment, thus an increase 112 in the gray level is depicted in the graph of FIG. 4. Next, the contrast agent gradually fills the segment until such time when the segment is saturated and a steady state 114 is achieved. At that time, the segment is fully colored by the contrast agent.

Turning now to FIG. 5 showing a family of graphs of the type shown in FIG. 4, on a common coordinate system. Each graph 116, 117, 118, 119 represents the changing gray level relatively to the time, at a different location along the artery segment, x. In the example shown, graph 119 depicts the part of the segment of the artery to which the contrast agent arrives initially, while graph 116 depicts the part of the segment to which the contrast agent arrives last.

Referring now to FIG. 6 showing an illustration of a top view of the three-dimensional collection of graphs 116, 117, 118, 119 of FIG. 5, where the gray level is presented by a pattern. The higher the gray level at FIG. 5, the denser the pattern is, at FIG. 6. It should be noted, that although the gray level at FIG. 6 are discrete, since FIG. 6 is exemplary only, the gray levels are actually continuous. The time immediately after the injection and before the contrast agent reaches a certain point in the artery is the no-contrast region 120 in FIG. 6. The transient region 124 represents the segment location where and time in which the contrast agent gradually fills the artery. The saturation area 128 represents the segment location where and time in which the whole relevant segment is filled with the contrast agent. The tangent of the angle α 132 between the major axis 136 of the transient region 124 and the time axis, represents the velocity of the blood flow. This method provides the velocity Si for the i-th time slot of the heart beat cycle.

Repeating the injection and the analysis for different time segments will provide the velocities {Si} for the full heart beat cycle.

The time interval between the injection of the contrast agent and the arrival of the agent to a certain location along the artery can be used for more accurate estimation of TIMI grade, by applying the velocity at multiple points method at one point, for bolus arrival time measurements.

Referring now back to FIG. 2, the average artery segment output calculating component receives the Si for the i-th time slot, and calculates the blood volume flowing through the artery segment during the i-th time slot ΔBᵢ, using the formula:

$$Δ Bi = \frac{Si * Δ t * Vi}{L}$$

Multiplying the velocity Si by the duration of the time slot Δt yields the distance traveled by the material during the time slot. Dividing this ratio by the length of the segment, L, yields the relative part of the segment traveled by the material. Multiplying this quantity by the volume of the artery segment at that point in time, yields the quantity of blood that flew through the segment during this time slot.

The average artery segment output component, 58, first determines the overall volume of blood flow through the artery segment during the heart beat cycle, B. B is determined as the summation of the volumes of the blood flows over all time slots:

$$B = {\sum }_{i = 1}^{n} Δ Bi$$

The average artery segment output, Q, is the total volume, divided by the duration of the heart beat cycle, T.

### Therefore

$$Q = \frac{B}{T}$$

If the overall volume of blood flow through the artery segment during the heart beat cycle, B, or the average artery segment output, Q, are of interest, their values are output by the average artery segment output component, 58.

The coronary reserve component 62 takes as input the average artery segment output in rest conditions, Qr, and the average artery segment output in hyperemia, Qh. The coronary reserve is the ratio:

$$CFR = \frac{Qh}{Qr}$$

In a preferred embodiment of the present invention. relative coronary reserve calculation component 66 takes as input the CFR of two arteries, CFRa which is the artery diseased or suspect of being diseased and CFRb which is of a healthy artery and calculates their ratio:

$$rCFR = \frac{CFRa}{CFRb}$$

It will be understood that if a healthy artery is not present than the relative coronary flow will not be calculated.

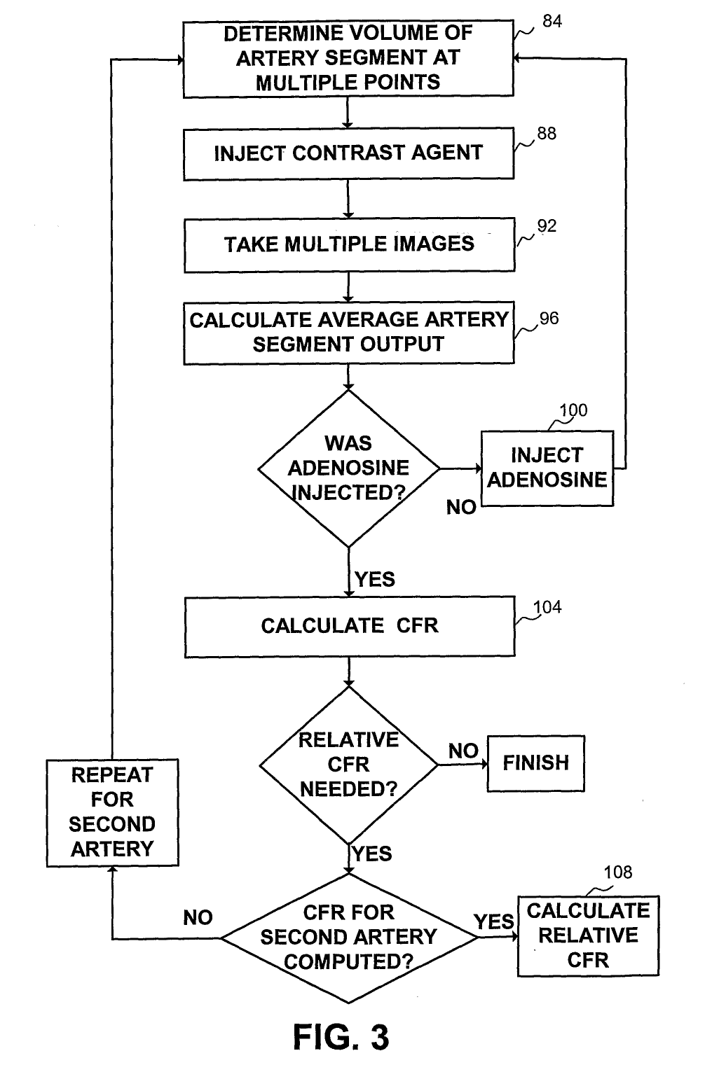

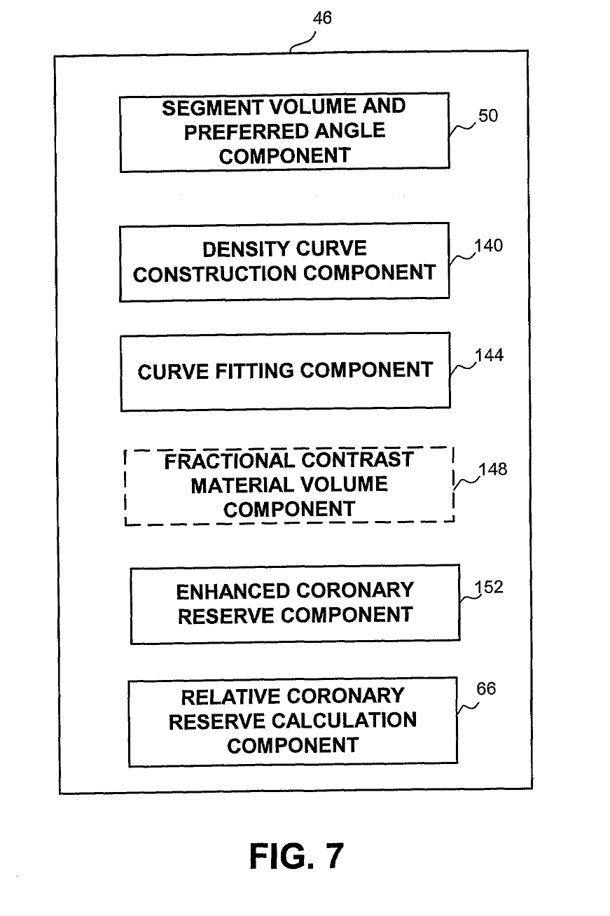

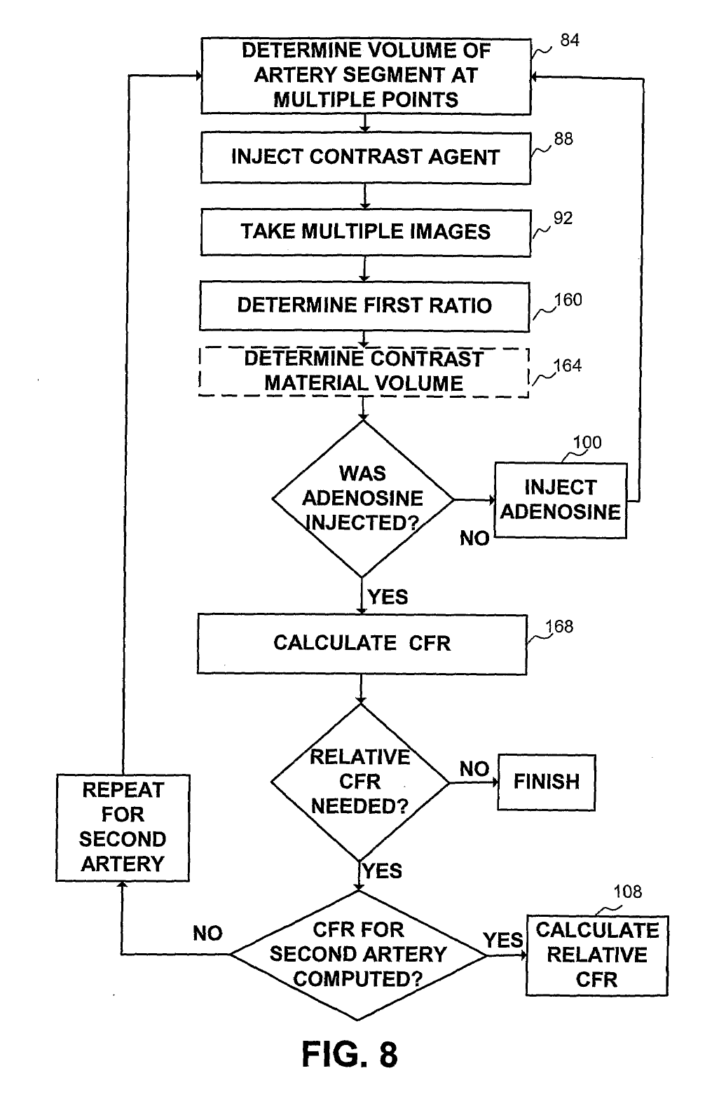

Referring now to FIG. 7 which shows an alternative to components 54, 56, 58, and 62 of FIG. 2, and to FIG. 8 which shows an alternative to step 96 of FIG. 3, by using the following analysis. Blood does not behave like a Newtonian fluid. Blood is a bi-phase material, containing plasma and blood cells, and therefore behaves like pseudoplastic material. In addition, due to the Farhaeus-Lindqvist effect, blood cells move faster than the plasma in the center of the is vessel, and since the coronary arteries are not long enough, the velocity profile is not fully developed. All these factors cause the blood velocity profile to be non-compatible with a parabolic model and there is no analytic way to describe all the behaviors simultaneously. Therefore, an empiric model for the blood velocity, developed by Verhoeven at 1985 that determines the blood flow is adopted. According to the model:

$$V ( r ) = \frac{Vm}{1 - \frac{1}{3} \cdot ln [ 1 - {( \frac{r}{R} )}^{2} ]} \qquad ( 1 )$$

Wherein:

R is the radius of the vessel;

r is the distance of the measured point from the center of the vessel;

Vₘ is the maximal velocity, which is the velocity at the center of the vessel; and

V(r) is the velocity at distance r from the center of the vessel.

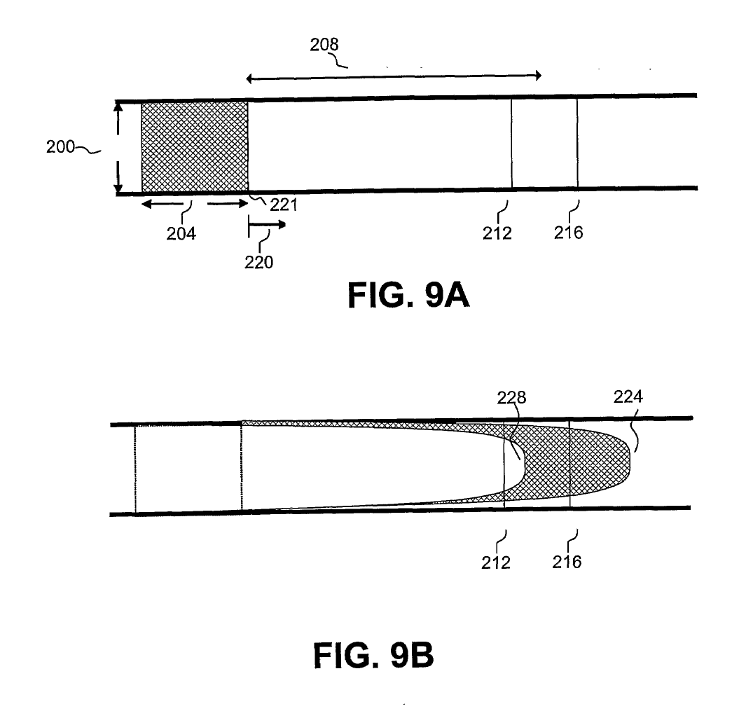

Referring now to FIG. 9A, showing an artery segment during contrast material injection, and FIG. 9B showing the same segment at time t after the injection. The contrast material is injected at area 204, denoted by L, and the injection point is considered to be the most distal point area 204, i.e. 221. The region of interest (ROI), for which the gray levels are measured resides between 212 denoted by X₁ and 216 denoted by X₂. Distance 208 is the mean distance between the injection point and the region of interest, and is denoted by X̅. The distance referred to as 200, is equal to 2*R. The direction along which the blood flows is denoted by x and referred to by arrow 220, which starts at the injection point 221. On FIG. 9B, the contrast material is shown to be dispersed as a bolus between two borderlines, the upper borderline denoted by 224 and the lower borderline denoted by 228. Upper borderline 224 and lower borderline 228 are determined using the velocity profile shown in (1).

Using image processing methods, including edge detection and gray level analysis performed over angiograms showing a cross section of the artery taken at multiple points in time during a heart beat cycle when the contrast material is dispersing in the artery segment, and the information related to the volume of the artery segment as obtained from the 3D model constructed earlier, the density values of the contrast material within the relevant artery segment are evaluated. Then a density curve, describing the percentage of the contrast material out of the total volume of the segment, is constructed from the density values. The density curve is built by density curve construction component 140 of FIG. 7, using the gray level analysis of the captured angiograms.

Geometrical analysis provides the volume bounded between upper borderline 224 and lower borderline 228 of FIG. 9B. By performing two reductions detailed below, the following formula for the volume of the contrast material present at time t within the ROI is obtained:

$${C}_{X 1 \div X 2} ( t ) = π {R}^{2} ( x 2 - x 1 ) \cdot [ 1 - {e}^{3} {e}^{\frac{- 3 Vm}{\overline{x}} \cdot t} ] \qquad ( 2 )$$

Wherein the applied reductions are as follows:

Dealing only with upper borderline 224 ignoring lower borderline 228.

Relating to X̅ instead of integrating between X₁ and X₂.

Equation (2) provides an approximated mathematical description of the density curve built from the angiograms. Therefore, the value of the plateau in the graph is equal to the constant πR²(x2−x1).

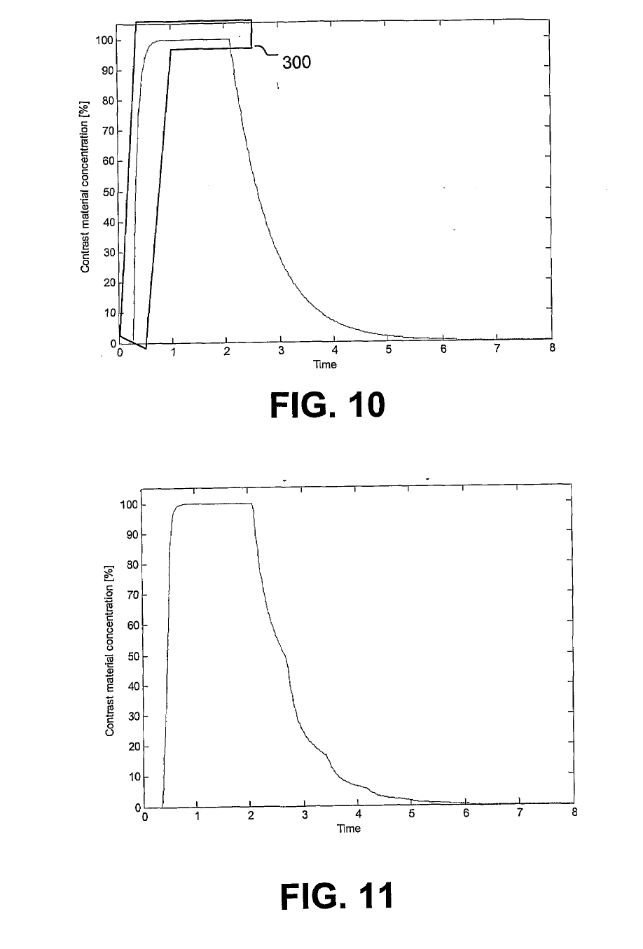

Referring now to FIG. 10, showing a density curve. By using curve fitting to the exponential area marked as 300, the exponential part of equation (2), i.e., −

$$\frac{{v}_{m}}{\overline{x}}$$

is obtained. The curve fitting is performed by curve fitting component 144 of FIG. 7. The same analysis applies also when the pulsatile nature of the blood flow as shown in FIG. 11. Depending on the nature of the curve, it may be beneficial, such as when considering the ascending and plateau parts of the density curve shown in FIG. 11 to perform the curve fitting to a logarithm of the density curve, instead of to the curve itself. The determination of said exponential part, being

$$\frac{{v}_{m}}{\overline{x}}$$

is performed at step 160 of FIG. 8. Once

$$\frac{{v}_{m}}{\overline{x}}$$

is obtained, Vr can be obtained for each r, and when integrated over the cross section and averaged over the region of interest, provides the flow through the artery at instant t, and when averaged over a full heart beat provides the average artery segment output. The determination of the ratio is performed at step 164 of FIG. 8, by fractional contrast material volume component 148 of FIG. 7. If the flow itself is of no importance, step 164 of FIG. 8 can be skipped and fractional contrast material volume component 148 of FIG. 7 can be omitted. In this case, when the only required information is the CFR, the CFR can be directly determined by

$$\frac{\frac{{v}_{m ( hypermeia )}}{\overline{x}} * CrossSection ( hyperemia )}{\frac{{v}_{m ( rest )}}{\overline{x}} * CrossSection ( rest )} ,$$

which is performed by enhanced coronary reserve component 152 of FIG. 7. If the cross section of the artery does not change between rest and hyperemia conditions, the ratio between

$$\frac{Vm}{\overline{x}}$$

at hyperemia and at rest provide the CFR. The CFR calculation is performed at step 168 of FIG. 8, which is different from step 104 of FIG. 3, since it determines the ratio between the two rations, rather than the ratio between two volumes, as in FIG. 3. As in FIG. 3 above, the relative CFR is determined at step 108 of FIG. 8, as the ratio between the CFR of a diseased artery and the CFR of a healthy artery.

The proposed steps have a number of advantages: The CFR can be found without determining X̅, by just keeping the same location of ROI and injection area; If the flow itself is required, then X̅ can be approximated by the center of the ROI; curve fitting is simple as one parameter only needs to be evaluated; and curve fitting eliminates noise and provides a more robust solution.

If the first reduction discussed above, of ignoring the lower borderline is to be avoided, the density graph comprises three parts: the ascending part, in which contrast material is arriving into the ROI, the part being described by the formula:

$${C}_{X 1 \div X 2} ( t ) = K \cdot [ 1 - {e}^{3} {e}^{\frac{- 3 Vm}{\overline{x}} \cdot t} ] \qquad ( 3 )$$

the middle part in which material is arriving and leaving the ROI, and is described by the formula:

$${C}_{X 1 \div X 2} ( t ) = K \cdot {e}^{3} \cdot [ {e}^{\frac{- 3 Vm}{\overline{x} + L} \cdot t} - {e}^{\frac{- 3 Vm}{\overline{x}} \cdot t} ] \qquad ( 4 )$$

and the third part in which the material is leaving the ROI, which is described by the formula:

$${C}_{X 1 \div X 2} ( t ) = K \cdot {e}^{3} \cdot {e}^{\frac{- 3 Vm}{\overline{x} + L} \cdot t} \qquad ( 5 )$$

Under these conditions, three curves are fitted in order to obtain the three parameters:

$$K , \frac{- 3 Vm}{\overline{x}} , \\ and \frac{- 3 Vm}{\overline{x} + L}$$

The disclosed method is not limited to the assumed Verhoeven velocity profile, which is merely an example, and to the

$$\frac{Vm}{\overline{x}}$$

parameter. Other velocity profiles can be used, and different parameters can be obtained from the graphs and used for determining the flow output or the arterial reserve. Furthermore, the method is not limited to one parameter, but can rather use any number of parameters and combinations thereof.

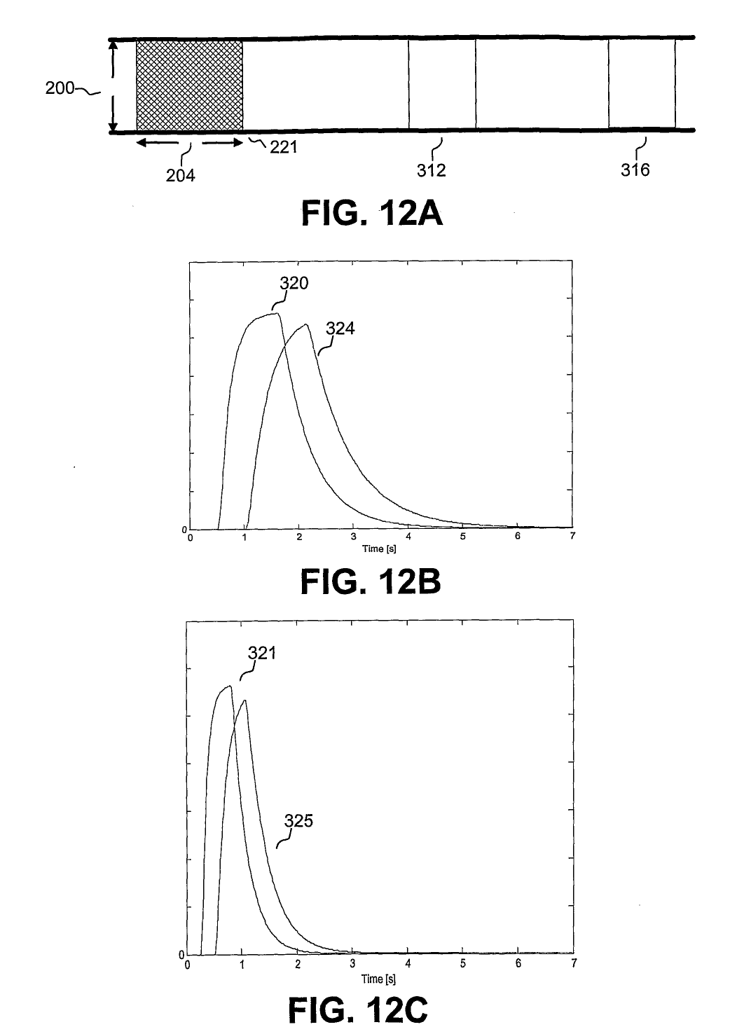

As yet another alternative to steps 54, 56, 58, and 62 of FIG. 2, a model of considering two ROIs, a proximal one and a distal one is disclosed. FIG. 12A shows a proximal ROI denoted by 312 and a distal ROI denoted by 316. FIG. 12B shows a density curve for proximal ROI 312, marked as 320 and a density curve for distal ROI 316 marked as 324, both density curves match the respective ROIs at resting, i.e., non-hyperemic conditions. FIG. 12C shows density curve 321 corresponding to proximal ROI 312 and density curve 325 corresponding to distal ROI 316, both at hyperemic conditions. A method known in the art for determining the CFR is by determining the ratio between: 1. the time shift between density curves 320 and 324, and 2. The time shift between density curves 321 and 325, is the CFR. The time shifts can be found, for example by cross-correlation between curves 320 and 324, and cross-correlation between curves 321 and 325. The disclosed method proposes an improved and more accurate alternative, being determining the CFR as the ratio between: 1. the time shift between density curves 320 and 321, and 2. the time shift between density curves 324 and 325.

In yet another alternative, the curves at FIG. 12B are shrunk, until maximal similarity between the shrunk curves of FIG. 12B and the curves of FIG. 12C is achieved. The shrinking factor is the CFR. Alternatively, the curves at FIG. 12C are stretched, until maximal similarity between the stretched curves of FIG. 12C and the curves of FIG. 12B is achieved. The maximal similarity can be obtained, for example by cross correlation.

The proposed methods can be further improved by employing a few enhancements to the injection procedure. A first enhancement comprises performing the injection when the flow in the artery is minimal, i.e., during the systole (this is true only for the coronary artery, unlike the rest of the body), which provides two advantages. The first advantage is that the shape of the contrast material bolus is more distinct when there is little or no flow around it, i.e., less external forces are applied to the material. The second advantage is that the contrast agent causes minimal interrupt to the measured flow. Another proposed enhancement is to inject the contrast material continuously throughout an integer number of heart beat cycles, i.e., a time period which is a product of an integer number and the duration of a heart beat cycles of the subject. Using this enhancement, the upper and the lower borderlines, as shown in FIG. 9B will be synchronized, i.e., they will occur at every point along the artery segment at exactly the same phase of the heart beat cycle. Yet another enhancement is to create a compact bolus of contrast material, by injecting the material radially, i.e., sideways towards the walls of the artery rather than from the tip of the catheter in the distal direction. The radial injection will significantly straighten the upper and lower borderlines, so that the relevant artery segment will assume a substantially cylindrical shape.

It will be appreciated by persons skilled in the art that various other methods and nuances of determining the velocity or the output of the artery segment from gray levels exist. The methods originate from different formulas representing the blood flow, either analytical or empirical, different analysis and reductions and different mathematical methods.

Referring now to FIG. 3, showing the flow diagram of the process of calculating the coronary reserve of an artery. In step 84 at least one pair of images of the artery are taken at multiple times throughout a heart beat cycle. In each pair, the two images are taken from different projection angles. From each pair of images, a three-dimensional model of the artery segment is generated, and the volume of a fixed part of the artery is determined by a relevant software program or part of a program, such as CardiOp made by Paieon from Rosh Ha'Ayin, Israel. The part of the artery of interest is defined, for example, by a fixed distance from a stenosis in the distal direction and a possibly different distance in the proximal direction. Instead of a stenosis, any other fixed point can be used as a reference point for the artery segment. Preferably, as long as possible, artery segments with no substantial branching are chosen. However, branches flow can be subtracted for accurate measurement and calculations when the artery segments do include branches. The fixed length can be anywhere from about a few millimeters to about 15 centimeters from the fixed point in the proximal and the distal directions. During step 84, the projection angle which is most perpendicular to the artery segment is calculated form the three dimensional model of the artery segment. Step 84 can be accomplished also by extrapolation of a 3D model performed at one point in time during the heart beat cycle to the entire heart cycle.

In step 88, contrast agent is injected to the artery through the use of the catheter as shown in FIG. 1 or through the use of other means such as a needle, intravenous tube and the like or a specific controlled injection system. In step 92 multiple images are taken from the projection angle calculated in step 84, throughout a heart beat cycle, immediately after the contrast agent injection. The images are taken at points in time corresponding to the times at which images were taken at step 84, i.e., at the same times relatively to the heart beat cycle. The more images taken during the heart beat cycle, the better is the resolution received and therefore the accuracy in providing the CFR and rCFR. The number of images taken depends on the duration of the heart beat cycle and on the image-acquiring rate of the imaging device. Determining the exact sequence of images taken throughout exactly one full heart beat cycle is automatically achieved by the proposed system, using data from the catheterization work station 15 of FIG. 1, or any other synchronization equipment. In step 96, the average artery segment output calculation throughout the heart beat cycle is determined using the fractional blood volume calculation component 54 of FIG. 2. and the average artery segment output calculation 58 of FIG. 2.

In step 100, a coronary vasodilator, such as adenosine, is injected to the subject, and steps 84, 88, 92, and 96 are repeated, so that the average artery segment output is calculated for the artery in hyperemia. Optionally, step 84 of determining the volume of the artery segment at multiple points can be skipped, and the volumes determined prior to the injection of the coronary vasodilator can be used also subsequent to the injection, taking into account the shortened heart beat cycle. This is possible since the coronary vasodilator mainly dilates the small vessels that infuse blood into the muscles rather than the large vessels whose volume does not change significantly. In addition, since the heart beat cycle is shortened due to the injection of coronal vasodilator, it is possible to ignore some of the volume values that were collected prior to the injection and use only a subset. Then, in step 104, the coronary reserve is calculated using the coronary reserve calculation component 92 of FIG. 2, by determining the ratio between the average artery segment flow output following the adenosine injection (i.e. in hyperemia) and the average artery segment flow output prior to the adenosine injection (in rest condition).

In order to determine the relative coronary reserve, steps 84, 88, 92, 96, 100, and 104 are repeated for a second artery segment, i.e., the CFR is determined for a second artery segment. Then, in step 108, the relative coronary reserve is determined by the ratio between the coronary reserves of the first and the second artery segments.

Persons skilled in the art will appreciate that in order to determine the CFR and the relative CFR with higher degree of precision, the described process can be performed over multiple heart beat cycles or parts thereof rather than one, thus increasing the averaging accuracy. Since the process averages the volume of blood flowing through an artery segment over time, the number of heart beat cycles considered in resting conditions and in hyperemia need not be equal. Similarly, the number of heart beat cycles considered for the diseased artery and for a healthy artery need not be equal as well.

The above shown examples serve merely to provide a clear understanding of the invention and not to limit the scope of the present invention or the claims appended thereto. Persons skilled in the art will appreciate that other variants of the method and systems can be used in association with the present invention so as to meet the invention's goals. Different methods of determining the volume of an artery segment, or of determining the average flow through an artery segment can be employed.

The presented method and apparatus are innovative in terms of using the volume and the flow information of the artery segment either for a specific time slice or throughout a full heart beat cycle with timing adjustments of the 3D model and volumes to the velocity measurements. The proposed invention yields the CFR and the relative CFR with higher degree of precision, without requiring a higher degree of invasiveness than a standard catheterization. The invention carries out the calculations based solely on angiograms, and does not require additional equipment or special expertise on the side of the physician, thus it is easy and cheap to employ.

The method is accurate, since the data concerning the volume of the artery and the velocity of the blood through the artery are collected independently, thus avoiding the interrelations between the factors. Furthermore, since the structure of the artery is found first, it enables the determination of optimal projection view to be used for the velocity determination stage in order to minimize the need for imaging conditions compensation. However, if such compensation is required, it is best determined once the artery's structure and orientation is known. The data for the stages is collected form the artery only, and not form other areas captured in the angiograms, thus minimizing undesired effects. The method is highly accurate also since the data is collected and analyzed separately for each frame throughout the heart beat cycle, but the total results take into account the information collected throughout the cycle. In addition, performing the gray level analysis over the whole artery segment, minimizes problems of measurement fluctuations.

It will be appreciated by persons skilled in the art that the present invention is not limited to what has been particularly shown and described hereinabove. Rather the scope of the present invention is defined only by the claims which follow.

1. A method for determining the arterial reserve of a subject having a blood flow, said blood flow having a velocity below or equal to a maximal velocity value, the method comprising the following steps:
receiving an at least one first model, said model representing an at least one substantially fixed segment of an at least one artery of the subject at a plurality of points in time associated with and during an at least one part of an at least one first heart beat cycle, said artery segment having a proximal cross section and a distal cross section;
injecting contrast agent into the at least one artery of the subject, said subject being in a non-hyperemic state, said contrast agent is injected at an injection area having a proximal cross section and having a distance from the at least one substantially fixed segment;
determining an at least one first parameter from an at least one first angiogram representing the substantially fixed artery segment, said first angiogram taken from a first projection angle;
injecting the subject with substance that simulates hyperemia;
receiving an at least one second model, representing the at least one substantially fixed segment of the at least one artery of the subject at a plurality of points in time associated with and during an at least one part of an at least one second heart beat cycle;
injecting the contrast agent to the at least one artery of the subject, said subject being in a hyperemic state, said contrast agent is injected at the injection area;
determining an at least one second parameter from an at least one second angiogram representing the substantially fixed segment, said second angiogram taken from a second projection angle; and
determining the arterial reserve as the ratio between one of any of the at least one first parameter or a combination thereof and any of the at least one second parameter or a combination thereof.

2. The method of claim 1 wherein determining the first parameter comprises the steps of:
determining from the at least one first angiogram, arterial segment output values of the blood flow within the at least one fixed segment of the at least one artery being in a non-hyperemic state, at times corresponding to the plurality of points in time associated with the at least one part of the at least one first heart beat cycle;
summing the arterial segment output values determined during the at least one part of the at least one first heart beat cycle, said arterial segment being in a non-hyperemic state; and
dividing the summed arterial segment output values by the duration of the at least one part of the at least one first heart beat cycle, yielding an average non-hyperemic artery segment flow output.

3. The method of claim 1 wherein determining the second parameter comprises the steps of:
determining from the at least one second angiogram, arterial segment output values of the blood flow within the at least one fixed segment of the at least one artery being in a hyperemic state, at times corresponding to the plurality of points in time associated with the at least one part of the at least one second heart beat cycle;
summing the arterial segment output values determined during the at least one part of the at least one second heart beat cycle, said arterial segment being in a hyperemic state; and
dividing the summed arterial segment output values by the duration of the at least one part of the at least one second heart beat cycle, yielding an average hyperemic artery segment flow output.

4. The method of claim 1 further comprising the step of determining a fractional velocity of the blood flow within the at least one fixed segment of the at least one artery, at times corresponding to the plurality of points in time associated with the at least one part of the at least one first heart beat cycle.

5. The method of claim 4 wherein the fractional velocity is determined by analyzing the whole artery segment for gray level changes in the artery.

6. The method of claim 4 wherein the fractional velocity is determined by analyzing local gray level curves in multiple points of the artery.

7. The method of claim 1 further wherein the first parameter is a ratio between the maximal velocity value of blood within the artery segment when the subject is at a non-hyperemic state and the distance between the substantially fixed segment and the injection area.

8. The method of claim 1 wherein the second parameter is a ratio between the maximal velocity value of blood within the artery segment when the subject is at a hyperemic state and the distance of the artery segment from the injection area.

9. The method of claim 1 wherein determination of the at least one first parameter comprises the steps of:
determining from the at least one first angiogram taken at a predetermined projection angle and the at least one first model, a density curve for the at least one artery segment;
obtaining a velocity profile of the blood flow within the at least one artery;
performing curve fitting for the density curve to determine the first parameter.

10. The method of claim 1 wherein determination of the at least one second parameter comprises the steps of:
determining from the at least one second angiogram taken at a predetermined projection angle and the at least one second model a density curve for the at least one artery segment;
obtaining a velocity profile of the blood flow within the at least one artery;
performing curve fitting for the density curve to determine the second parameter.

11. The method of claim 1 wherein the distance between the injection area and the at least one substantially fixed segment is the distance between the distal cross section of the injection area and a cross section of the substantially fixed artery segment located at equal distances from the proximal cross section and from the distal cross section of the substantially fixed artery segment.

12. The method of claim 1 further comprising the step of creating the first or the second models of the at least one artery of the subject.

13. The method of claim 1 wherein the first or the second models are three dimensional models.

14. The method of claim 1 further comprising the step of determining the projection angle and the volumes of the at least one substantially fixed segment of the at least one artery of the subject at a plurality of points in time associated with and during the at least one part of the at least one first heart beat cycle, from the first model.

15. The method of claim 1 further comprising the step of determining the projection angle and the volumes of the at least one substantially fixed segment of the at least one artery of the subject at a plurality of points in time associated with and during the at least one part of the at least one second heart beat cycle, from the second model.

16. The method of claim 1 further comprising the step of compensating for the non-perpendicularity of the at least one substantially fixed segment of the at least one artery of the subject.

17. The method of claim 1 further comprising the step of registering the at least one first angiogram with the first model.

18. The method of claim 1 further comprising the step of registering the at least one second angiogram with the second model.

19. The method of claim 1 further comprising a step of determining TIMI grades from local gray level curves in multiple points of the artery.

20. The method of claim 1
further comprising the step of determining a relative arterial reserve as the ratio between the arterial reserve determined for a first artery segment and the arterial reserve determined for a second artery segment.

21. The method of claim 20 wherein the first artery segment is diseased or is suspect as being diseased and the second artery segment is healthy.

22. The method of claim 1 wherein the arterial reserve is an arterial coronary reserve.

23. The method of claim 1 wherein the contrast agent injection is performed during the systole of the subject.

24. The method of claim 1 wherein the contrast agent injection is performed continuously throughout an integer number of heart beat cycles of the subject.

25. The method of claim 1 wherein the contrast agent is injected radially.

26. A method for determining the blood flow output of a subject, the method comprising the following steps:
receiving at least one model, representing an at least one substantially fixed segment of an at least one artery of the subject at a plurality of points in time associated with and during an at least one part of an at least one heart beat cycle, said artery segment having a proximal cross section and a distal cross section;
injecting contrast agent into the at least one artery of the subject, said contrast agent is delivered to the at least one fixed segment; and
determining from an at least one angiogram taken at a predetermined projection angle, arterial segment output values of the blood flow within the at least one fixed segment of the at least one artery, at times corresponding to the plurality of points in time associated with the at least one part of the at least one heart beat cycle.

27. The method of claim 26, further comprising the steps of
summing the arterial segment flow output values determined during the at least one part of the at least one heart beat cycle; and
dividing the summed arterial segment flow output values by the duration of the at least one part of the at least one heart beat cycle, yielding an average artery segment flow output.

28. The method of claim 26, wherein determining the arterial segment output values comprises the steps of:
determining from the at least one angiogram and the at least one model, a density curve for the substantially fixed segment;
obtaining a velocity profile of the blood flow within the at least one substantially fixed segment:
performing curve fitting for the density curve to determine an at least one parameter;
substituting the at least one parameter in the velocity profile to determine velocity values; and
integrating the velocity values over a cross section of the substantially fixed segment to obtain the arterial segment output values of the blood flow within the at least one substantially fixed segment of the at least one artery.

29. The method of claim 26, wherein the parameter is ratio between the maximal velocity of blood within the substantially fixed segment and the distance of the at least one substantially fixed segment from the injection point.

30. The method of claim 26 wherein the distance between the injection area and the at least one substantially fixed segment is the distance between the distal cross section of the injection area and a cross section of the at least one substantially fixed artery segment located at equal distances from the proximal cross section and from the distal cross section of the at least one substantially fixed artery segment.

31. The method of claim 26 further comprising the step of creating the at least one model of the at least one substantially fixed segment of the at least one artery of the subject.

32. The method of claim 26 further comprising the step of determining the projection angle from the at least one model.

33. The method of claim 26 further comprising the step of compensating for the non-perpendicularity of the at least one substantially fixed segment of the at least one artery of the subject.

34. The method of claim 26 further comprising the step of registering the angiograms with the at least one model.

35. The method of claim 26 wherein the at least one model is a three-dimensional model.

36. The method of claim 26 further comprising a step of determining TIMI grades from local gray level curves in multiple points of the artery.

37. The method of claim 26 further comprising the step of determining the fractional velocity of the blood flow within the at least one fixed segment of the at least one artery, at times corresponding to the plurality of points in time associated with the at least one part of the at least one heart beat cycle.

38. The method of claim 37 wherein the fractional velocity is determined by analyzing the whole artery segment for gray level changes in the artery.

39. The method of claim 37 wherein the fractional velocity is determined by analyzing local gray level curves in multiple points of the artery.

40. A method for determining the arterial reserve of a subject, the method comprising the following steps:
receiving an at least one first model, representing at least two substantially fixed segments of an at least one artery of the subject at a plurality of points in time associated with and during an at least one part of an at least one first heart beat cycle;
injecting contrast agent into the at least one artery of the subject, said contrast agent is delivered to the at least two substantially fixed segments being in a non-hyperemic state;
determining from an at least one first angiogram taken at a predetermined projection angle, a first set of density curves, one density curve for each of the at least two substantially fixed segments;
injecting the subject with substance that simulates hyperemia;
receiving at least one second model, representing the at least two substantially fixed segments of the at least one artery being in a hyperemic state, at times corresponding to a plurality of points in time associated with an at least one part of an at least one second heart beat cycle;
injecting the contrast agent to the at least one artery of the subject, said contrast agent is delivered to the at least two substantially fixed segments being in a hyperemic state;
determining from an at least one second angiogram taken at a predetermined projection angle, a second set of density curves, one density curve for each of the at least two substantially fixed segment.

41. The method of claim 40 further comprising the steps of:
determining a first time shift between a first density curve taken from the first set of density curves and a second density curve taken from the second set of density curves, the first and the second density curves corresponding to a first substantially fixed segment;
determining a second time shift between a third density curve taken from the first set of density curves and a fourth density curve taken from the second set of density curves, the third and the fourth density curves corresponding to the second substantially fixed segment; and
determining the arterial reserve as the ratio between the first time shift and the second time shift.

42. The method of claim 40 further comprising the steps of:
shrinking the first set of density curves in a shrinking factor so that maximal similarity occurs between the first set being shrunk and the second set; and
determining the arterial reserve as the shrinking factor.

43. The method of claim 40 further comprising the steps of:
stretching the second set of density curves in a stretching factor so that maximal similarity occurs between the first set and the stretched second set; and
determining the arterial reserve as the stretching factor.

44. A computing platform for determining the arterial reserve of a subject from at least two images taken at a projection angle, the computing platform executing:
a component for receiving a model of an at least one substantially fixed segment of an at least one artery, said segment having a volume;
a gray level extraction component for extracting gray level representing the material filling rate and diminishing rate along the at least one artery;
a parameter determination component for determining an at least one parameter associated with the blood flow through the at least one substantially fixed segment of an at least one artery; and
an enhanced artery reserve component for determining the arterial reserve as a ratio between a first parameter associated with a first density curve associated with the substantially fixed segment being in a hyperemic state and a second parameter associated with a second density curve associated the substantially fixed segment being in a non-hyperemic state.

45. The computing platform of claim 44 wherein the parameter determination component comprises:
a density curve construction component for constructing a density curve associated with the at least one substantially fixed segment from the gray levels; and
a curve fitting component for determining a parameter associated with the density curve or a part thereof.

46. The computing platform of claim 44 wherein the parameter determination component comprises:
a fractional blood volume component for determining a fraction of the volume of the blood flow along the at least one artery at a plurality of points in time associated with and during an at least one part of an at least one heart beat cycle; and
an average artery segment output component for determining the average artery segment output during the at least one part of the at least one heart beat cycle of the at least one artery.

47. The computing platform of claim 44 further comprising a segment volume component for determining the projection angle or the volume of the at least one substantially fixed segment of an at least one artery from the model.

48. The computing platform of claim 44 further comprising a relative arterial reserve component for determining the relative arterial reserve between the first artery and a second artery, said relative arterial reserve being the ratio between the arterial reserve of the first artery and the arterial reserve of the second artery.

49. The computing platform of claim 44 further comprising at least one image acquiring device.

50. The computer platform of claim 44 further comprising a device for transferring images acquired by an image acquiring device to a processing unit, the processing unit comprises an at least one input and output devices for receiving input and presenting output to a user.

51. The computing platform of claim 44 further comprising a storage device for storing the images or the determined arterial reserve values.

52. The computing platform of claim 44 further comprising a segment volume component for determining the projection angle and the volume of an at least one segment of an at least one artery from the model.

53. A computing platform for determining the arterial flow of a subject from at least two images, the computing platform executing:
a component for receiving a model and volumes of an at least one substantially fixed segment of an at least one artery;
a gray level extraction component for extracting gray level representing the material filling rate and diminishing rate along the at least one artery;
a density curve construction component for constructing a density curve from the gray levels;
a parameter determination component for determining an at least one parameter associated with the blood flow through the at least one substantially fixed segment of an at least one artery; and
a curve fitting component for fitting a curve to the density curve or a part thereof; and
a fractional contrast material volume component for determining the artery segment output during an at least one part of an at least one heart beat cycle of the at least one artery.

54. The computing platform of claim 53 wherein the parameter determination component comprises:
a density curve construction component for constructing a density curve associated with the at least one substantially fixed segment from the gray levels and
a curve fitting component for determining a parameter associated with the density curve or a part thereof.

55. The computing platform of claim 53 wherein the parameter determination component comprises:
a fractional blood volume component for determining a fraction of the volume of the blood flow along the at least one artery at a plurality of points in time associated with and during an at least one part of an at least one heart beat cycle; and
an average artery segment output component for determining the average artery segment output during the at least one part of the at least one heart beat cycle of the at least one artery.

56. The computing platform of claim 53 further comprising a segment volume component for determining the volume of an at least one substantially fixed segment of an at least one artery.
